(19) United States
(12) Patent Application Publication
(10) Pub. No.: US 2009/0275824 A1
(43) Pub. Date: Nov. 5, 2009

(54) System and Method for Sensing Gastric Contractions

(75) Inventor: Martin Cholette, Acton, CA (US)

Correspondence Address: PACESETTER, INC., 15900 VALLEY VIEW COURT, SYLMAR, CA, 91392-9221, US

(73) Assignee: PACESETTER, INC., Sylmar, CA (US)

(21) Appl. No.: 12/112,930

(22) Filed: Apr. 30, 2008

(51) Int. Cl. A61B 5/055; A61B 5/103

(52) U.S. Cl. 600/424; 600/595

(57) ABSTRACT

An exemplary implantable device includes an emitter to emit radiation to illuminate a portion of the stomach and a detector to detect emitted radiation reflected by the portion of the stomach where a contraction of the stomach alters the reflected radiation. For example, during contraction, blood is excluded from the contracting region of the stomach and the stomach becomes less red in color. An exemplary method includes illuminating a portion of the gastrointestinal tract, detecting a change in illumination received by a detector where the change in illumination is responsive to a contraction of the gastrointestinal tract. Various other methods, devices, systems, etc., are also disclosed.

### TECHNICAL FIELD

Exemplary methods, devices, systems, etc., presented herein generally relate to sensing gastric contractions.

### BACKGROUND

The digestive system plays a role in a variety of endocrine disorders and metabolic disorders (e.g., anorexia, obesity, gastroparesis, diabetes, etc.). As these disorders involve complex mechanisms, medicine often uses an interdisciplinary approach. For example, a patient with anorexia may be treated by a psychiatrist and an endocrinologist and a patient with morbid obesity may be treated by a surgeon (e.g., Roux-en-Y gastric bypass). Over the past 20 years, gastric electrical stimulation or gastric pacing has emerged as another option to treat obesity.

With respect to obesity, the number of patients undergoing bariatric surgery for the treatment of obesity, and the proportion of the health care budget dedicated to this health problem, is growing exponentially. Yet some believe that, as a public health measure, bariatric surgery in the United States is being pursued in a less than optimal manner. This belief supports further testing of more controllable treatment options that rely on implantable programmable electronic stimulation devices. In particular, implantable devices that deliver gastric electrical stimulation have been effective in normalizing gastric dysrhythmia, accelerating gastric emptying and improving nausea and vomiting.

Gastric electrical stimulation (GES) therapies generally aim to control gastrointestinal motility. For example, GES using short duration pulses can reduce nausea and vomiting in patients with gastroparesis and GES using longer duration pulses can pace intrinsic gastric slow waves and thus normalize gastric dysrhythmia. Electrical stimulation of the small intestine, colon, or anal sphincter has also been reported for the treatment of dumping syndrome, constipation, and fecal incontinency.

For treatment of obesity, a therapy known as reverse (or retrograde) gastric pacing (RGP) can impair intrinsic gastric myoelectrical activity and substantially and acutely reduce food intake. RGP with a tachygastrial frequency of 9 cycles/min delivered using a pair of submucosal gastric electrodes implanted 5 cm above the pylorus in human subjects resulted in a reduction in the consumption of water, a reduction in food intake and an increase in gastric retention of a solid meal. Reduced food intake and freedom from symptoms resulting from moderate gastric stimulation are indicative of the therapeutic potential of RGP in treating obesity.

A particular form of gastric electrical stimulation is sometimes referred to as tachygastrial electrical stimulation (TES) where tachygastrial frequencies induce tachygastria and reduce normal slow waves. TES delivered at the distal antrum can reduce food intake, in a manner likely attributed to TES-induced reduction in proximal gastric tone, gastric accommodation, antral contractility and gastric emptying.

As various disorders and therapies pertain to gastric motility and, more specifically, waves or contractions of the gastrointestinal tract, techniques to monitor GI physiology are useful. Various techniques are discussed herein for monitoring GI contractions as well as other GI physiology.

### SUMMARY

An exemplary implantable device includes an emitter to emit radiation to illuminate a portion of the stomach and a detector to detect emitted radiation reflected by the portion of the stomach where a contraction of the stomach alters the reflected radiation. For example, during contraction, blood is excluded from the contracting region of the stomach and the stomach becomes less red in color. An exemplary method includes illuminating a portion of the gastrointestinal tract, detecting a change in illumination received by a detector where the change in illumination is responsive to a contraction of the gastrointestinal tract. Various other methods, devices, systems, etc., are also disclosed.

### BRIEF DESCRIPTION OF THE DRAWINGS

Features and advantages of the described implementations can be more readily understood by reference to the following description taken in conjunction with the accompanying drawings.

FIG. 1 is a block diagram of a sequence of digestive phases.

FIG. 2 is a plot of some physiologic measures for digestion and a diagram of a portion of the gastrointestinal tract in a relaxed state and in a contracted state.

FIG. 3 is a series diagrams including a diagram for a sensor for sensing VO₂, a diagram for a sensor for sensing contractions and a diagram for an exemplary circuit for sensing contractions.

FIG. 4 is an approximate anatomical diagram that includes various portions of the gastrointestinal tract (e.g., the stomach, the duodenum and the intestines).

FIG. 5 is a diagram of a portion of the gastrointestinal tract.

FIG. 6 is a diagram of exemplary sensor locations shown with respect to the portion of the gastrointestinal tract of FIG. 5.

FIG. 7 is a diagram of an exemplary arrangement for acquiring one or more physiologic measures related to digestion.

FIG. 8 is a block diagram of an exemplary device for acquiring contraction information and for delivering stimulation to the gastrointestinal tract.

FIG. 9 is a block diagram of an exemplary method for adjusting one or more stimulation parameters for stimulating the gastrointestinal tract and an exemplary method for assessing capture.

FIG. 10 is a block diagram of an exemplary method for detecting contractions of the gastrointestinal tract and for calling for an action (e.g., related to a gastrointestinal related therapy).

### DETAILED DESCRIPTION

The following description includes the best mode presently contemplated for practicing the described implementations. This description is not to be taken in a limiting sense, but rather is made merely for the purpose of describing the general principles of the implementations. The scope of the described implementations should be ascertained with reference to the issued claims.

### Overview

Various exemplary devices, methods, systems, etc., described herein relate to measurement of gastrointestinal contractions and optionally one or more other gastrointestinal physiologic measures. A particular device includes an emitter to emit radiation and a detector to detect emitted radiation as reflected or transmitted by the gastrointestinal tract. Such a device can output a signal that varies as the gastrointestinal tract contracts and relaxes. The signal can be used for any of a variety of purposes. For example, the signal can provide feedback to a stimulation device, the signal can determine digestive phase (fasting, cephalic, gastric, intestinal, etc.), or the signal can trigger any of a variety of actions (e.g., patient alert, information transmission, etc.).

Digestive phases are discussed below followed by a description of some gastrointestinal physiologic measures. Exemplary devices, systems and methods are described that can acquire one or more gastrointestinal physiologic measures, which, in turn, may be used for any of a variety of purposes.

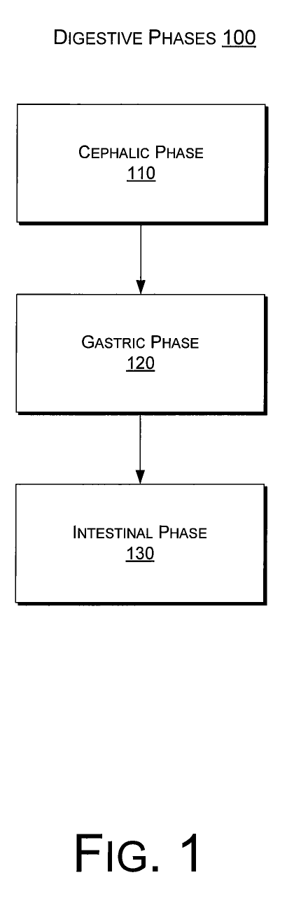

FIG. 1 shows a diagram of three digestive phases 100. Specifically, in a cephalic phase 110, the brain alerts the stomach that it should expect arrival of a meal and the stomach comes out of its interdigestive quiescence and begins low level motor and secretory activity. After a meal is consumed, in a gastric phase 120, the gastric motor and secretory activity increase significantly. If the meal is at all substantial, the gastric phase 120 is periodically suppressed by signals from the small intestine or by signals generated by the stomach if gastric pH falls to very low levels. Eventually, the meal is fully liquefied and emptied to the intestine (intestinal phase 130), and the stomach falls back into a state of very low motor and secretory activity, where it remains until the next cephalic phase 110.

More specifically, the cephalic phase 110 involves seeing, smelling and anticipating food and the brain informing the stomach that it should prepare for receipt of a meal. Communication is composed of parasympathetic stimuli transmitted through the vagus nerve to the enteric nervous system, resulting in release of acetylcholine in the vicinity of G cells and parietal cells. Binding of acetylcholine to its receptor on G cells induces secretion of the hormone gastrin, which, in concert with acetylcholine and histamine, stimulates parietal cells to secrete small amounts of acid. Additionally, a low level of gastric motility is induced. In essence, the gastric motor is turned on and begins to idle.

In the gastric phase 120, when a meal enters the stomach several additional factors come into play, foremost among them distension and mucosal irritation. Distension excites stretch receptors and irritation activates chemoreceptors in the mucosa. These events are sensed by enteric neurons, which secrete additional acetylcholine, further stimulating both G cells and parietal cells; gastrin from the G cells feeds back to the parietal cells, stimulating it even further. Additionally, activation of the enteric nervous system and release of gastrin cause vigorous smooth muscle contractions. The net result is that secretory and motor functions of the stomach are fully turned on—lots of acid and pepsinogen are secreted, pepsinogen is converted into pepsin and vigorous grinding and mixing contractions take place. However, there is a mechanism in place in the stomach to prevent excessive acid secretion: if lumenal pH drops low enough (less than about 2), motility and secretion are temporarily suspended.

In the intestinal phase 130, as food is liquefied in the stomach, it is emptied into the small intestine. It seems to be important for the small intestine to be able to slow down gastric emptying, probably to allow it time to neutralize the acid and efficiently absorb incoming nutrients. Hence, this phase of gastric function is dominated by the small intestine sending inhibitory signals to the stomach to slow secretion and motility. Two types of signals are used: nervous and endocrine. Distension of the small intestine, as well as chemical and osmotic irritation of the mucosa is transduced into gastric-inhibitory impulses in the enteric nervous system—this nervous pathway is called the enterogastric reflex. Secondly, enteric hormones such as cholecystokinin and secretin are released from cells in the small intestine and contribute to suppression of gastric activity.

Collectively, enteric hormones and the enterogastric reflex put a strong brake on gastric secretion and motility. As the ingesta in the small intestine is processed, these stimuli diminish, the damper on the stomach is released, and its secretory and motor activities resume.

It may be appreciated that the above mentioned digestive phases 100 include many sub-mechanisms. For example, mastication, saliva excretion, deglutition (swallowing), etc., are all part of the digestive process. Any of a variety of such mechanisms can trigger or condition gastrointestinal activity, which may alter gastrointestinal physiology.

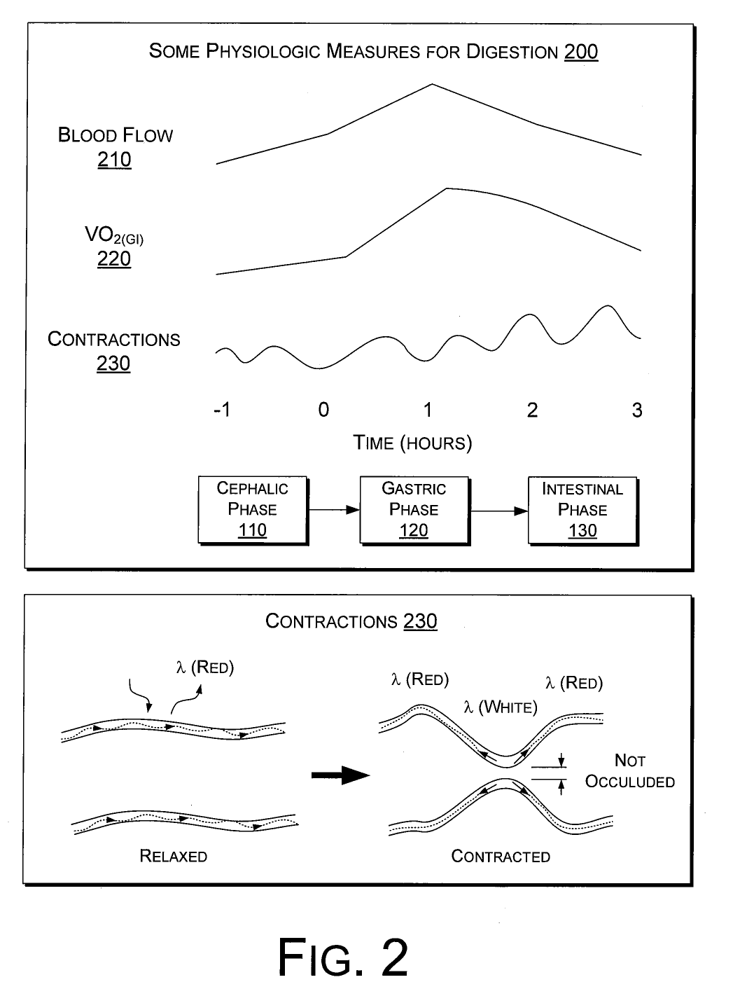

FIG. 2 shows a diagram of physiologic measures associated with digestion 200 along with a simplified diagram of gastrointestinal contractions 230. The measures include blood flow 210, VO₂(GI) 220 and contractions 230. Variations in these measures occur over time and may be associated with specific digestive phases 110,120 and 130. For example, blood flow 210, VO₂(GI) 220 and contractions 230 may commence at some low level during the cephalic phase 110. As food intake occurs (time 0 hours), then blood flow 210 increases to a peak value at about 1 hour after intake. VO₂(GI) 220 also increases and peaks around the same time as blood flow 210. Contractions 230 occur to assist in movement of food through the digestive tract.

As indicated by blood flow 210 and VO₂(GI) 220, the cardiovascular system responds to feeding. During the cephalic phase 110 and the initial ingestion of food, a transient increase in cardiac output, aortic blood pressure and heart rate occur, which may be accompanied by an increase in mesenteric vascular resistance. Within about 5 minutes to 30 minutes after feeding, cardiac output, heart rate and blood pressure return to normal while blood flow through the superior mesenteric artery begins to rise and continues to do so for about 30 minutes to about 90 min. These responses can be attenuated by administration of sympathetic blocking agents. Thus, as in the case of early postprandial thermogenesis, the secretion of catecholamines may play an important role in the cardiovascular changes that occur during this ingestion phase.

Alterations in blood flow 210 to the absorptive site concomitant with changes in gut motility (e.g., contractions 230) can influence the net absorption of nutrients such as amino acids and electrolytes. A positive correlation exists between blood flow 210 and the absorption of passively and actively transported substances. Increased blood flow 210 can increase absorption by increasing O₂ 220 delivery to the mucosa, altering tissue colloid osmotic pressure or increasing the removal of an absorbed nutrient, thus increasing the concentration gradient between the lumen and the blood. For example, absorption of amino acids from the jejunum was found to be directly proportional to blood flow 210 and inversely proportional to gut motility (e.g., contractions 230).

With respect to contractions 230, the gastrointestinal tract is highly vascularized. Oxygenated blood is red; thus, in a relaxed state, the gastrointestinal tract appears red. However, when a contraction occurs, blood is forced from the constricted region of the gastrointestinal tract and it appears white (i.e., less red) compared to relaxed portions. Such a change has an analogy in the term “white knuckles” where a driver may grip a steering wheel in a manner that constricts blood flow to the tissue around the knuckles of the hand. For the stomach, a contraction may travel as a band having a width of about a centimeter or two. Hence, a change in color occurs in association with the contraction. As described herein, various techniques measure this color change.

In FIG. 2, blood flow 210, VO₂ 220 and contractions 230 are shown together as relationships can exist between these measures. A study of blood flow in the left gastric artery in a canine model found that, in a fasting state, gastric motility and secretion exhibited periodical changes with an average cycle interval of 115.4±9.7 minutes (Naruse et al., “Interdigestive gastric blood flow: the relation to motor and secretory activities in conscious dogs”, Experimental Physiology, 77(5), 701-708). During a quiescent period, when gastric motility and secretion were minimal, the mean blood flow was stable at 33.9±3.8 ml/minute. During a contracting phase each peristaltic contraction was coupled with a rapid fall and rise in blood flow (from 10.5±1.9 ml/minutes below to 21.2±3.8 ml/minutes above the precontraction levels) in about 20 seconds to about 30 seconds. In addition there was a sustained elevation in blood flow (58.6±6.4 ml/minutes at the peak) lasting for 29.1±2.8 minutes. This study found that the onset of sustained blood flow elevation was preceded by that of motility in 63% of the cycles and that in 23% of the cycles, blood flow started to rise before the contracting phase began. Pepsin peaks coincided with blood flow peaks in two subjects and preceded the latter in the others. Feeding abolished periodic increases in motility and blood flow. The study concluded that left gastric arterial blood flow is not steady but exhibits dynamic changes in phase with periodic motor and secretory activities of the stomach in fasting conscious canines.

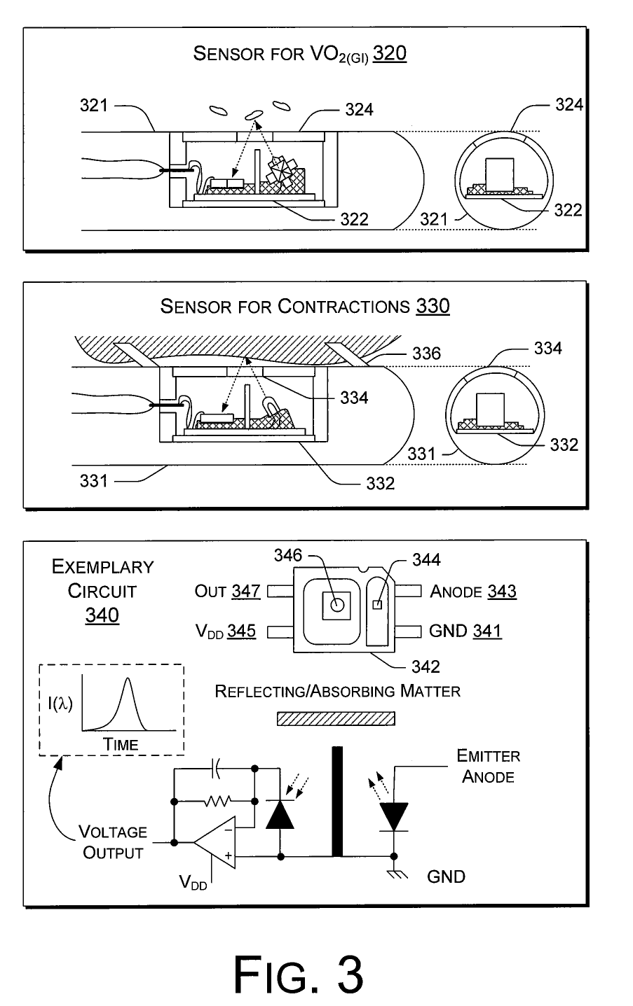

FIG. 3 shows a sensor for VO₂ measurement 320, a sensor for contraction measurement 330 and an exemplary circuit 340. As described herein, the sensor for VO₂ 320 can be used to measure contractions. However, other circuits can be used to measure contraction as well (e.g., exemplary circuit 340).

The sensor for VO₂ measurement 320 includes a lead body 321, sensor circuitry 322 and a window 324 that allows emitted radiation to interact with the surrounding environment and for reflected radiation to be detected. The particular example of FIG. 3 is described in detail in U.S. patent application Ser. No. 11/231,555, entitled “Implantable multi-wavelength oximeter sensor”, filed Sep. 20, 2005 and U.S. patent application Ser. No. 11/282,198, entitled “Implantable self-calibrating optical sensors”, filed Nov. 11, 2005, which are incorporated by reference herein.

The sensor for VO₂ 320 can include a beam combiner subassembly built into the sensor assembly where the sensor 322, in turn, is built into an implantable lead 321. The sensor circuitry 322 includes a photo detector and an optional application specific integrated circuit (ASIC). The lead 321 includes a compartment with a pair of end caps that can be used to hermetically seal the sensor circuitry 322. The lead 321 tube can be made of an opaque material, such as metal (e.g., titanium or stainless steel) or ceramic, so long as it includes a window 324 to allow for interaction with the surrounding environment. The window 324 can, for example, be made of synthetic sapphire or some other appropriate material. Exemplary synthetic sapphires are marketed by Imetra, Inc. (Elmsford, N.Y.) and Swiss Jewel (Philadelphia, Pa.).

The optional ASIC, which can include filters, analog-to-digital circuitry, multiplexing circuitry, and the like, controls the emitters and processes photo detector signals produced by a photo detector in any manner well known in the art. As described herein, an ASIC may provide signals indicative of the photo detector signals to an implantable device, such as an implantable monitor, pacemaker, ICD, and/or metabolic therapy device (e.g., to treat obesity, diabetes, etc.). If an ASIC or equivalent circuitry is not included within the sensor circuitry 322, and the sensor circuitry 322 outputs analog signals, such signals can be delivered between the sensor circuitry 322 and the implantable device.

In the sensor circuitry 322, an opaque optical wall is positioned between the beam combiner subassembly and the photo detector. The various components can be attached to a substrate (e.g., by epoxy) where the substrate can be a printed circuit board (PCB). Bond wires can be used to attach the various components to the substrate, as well as to attach the substrate to terminals which extend through an insulated feedthrough in an end cap of the sensor compartment.

With respect to a lead-based sensor, the size of the beam combiner is preferably about 2 millimeters (mm) or less, and the size of the sensor circuitry 322 is about 4 mm or less, and preferably about 3 mm. The length of the sensor circuitry 322, which extends axially in the lead 321 can be somewhat larger, because the length of the lead 321 is relatively large as compared to the diameter of the lead.

The lead 321 may have a transparent housing, or include its own window, opening, or the like. The lead 321 can include tines for attaching the lead 321 to a desired position; the lead 321 may include any of a variety of types of fixation means, or none at all. Additionally, the lead 321 may also include a lumen for a stylet, which can be used for guiding the lead to its desired position.

The sensor 320 can include wires that provide power and possibly control signals to the sensor circuitry 322 from an implantable device, and provide pulse oximetry signals from the sensor circuitry 322 to the implantable device.

As described herein, a lead may include one or more electrodes configured for delivery of energy. For example, the lead 321 may include a ring electrode and a tip electrode that are connected to an implantable device by way of wires.

The sensor for contractions 330 includes various features of the sensor for VO₂ 320. For example, the sensor 330 includes a lead 331, sensor circuitry 332 and a window 334. In the example of FIG. 3, a pair of tines 336 is also shown as being capable of anchoring the lead to tissue (e.g., stomach). The sensor circuitry 332 can differ from the circuitry 322 in that emitter requirements differ. More particularly, to sense a color change, as explained with respect to FIG. 2, the beam combiner subassembly is not necessarily required. Instead, a single emitter that emits a single wavelength or a band of wavelengths may be used.

The exemplary circuit 340 includes various features of the TRS series reflective color sensors marketed by TAOS (Texas, USA). The TRS series devices are configured to convert reflected light intensity to an output voltage that is directly proportional to the reflected light intensity on the photodiode. The devices include an integral color LED and a matching color filters on the photodiode. Various components of the TRS series device are configured as a monolithic silicon IC (e.g., photodiode, operational amplifier and feedback components). Colors include red (630 nm), green (567 nm) and blue (470 nm). The TRS series devices can be configured to be triggered such that the emitter emits radiation only when desired. Thus, an exemplary circuit may be pulsed at particular times or in response to certain events to measure reflected radiation, for example, to measure contractions of the gastrointestinal tract.

The circuit 340 includes a housing or package 342 that houses an emitter 344 and a detector 346. The housing 342 includes connection tabs ground 342, anode 343, supply voltage 345 and output voltage 347 for the internal circuitry, as indicated in the circuit diagram. The output voltage 347 corresponds to intensity versus time. Thus, for a sensor mounted adjacent the stomach, a contraction will cause the intensity to increase as the stomach turns from red to white.

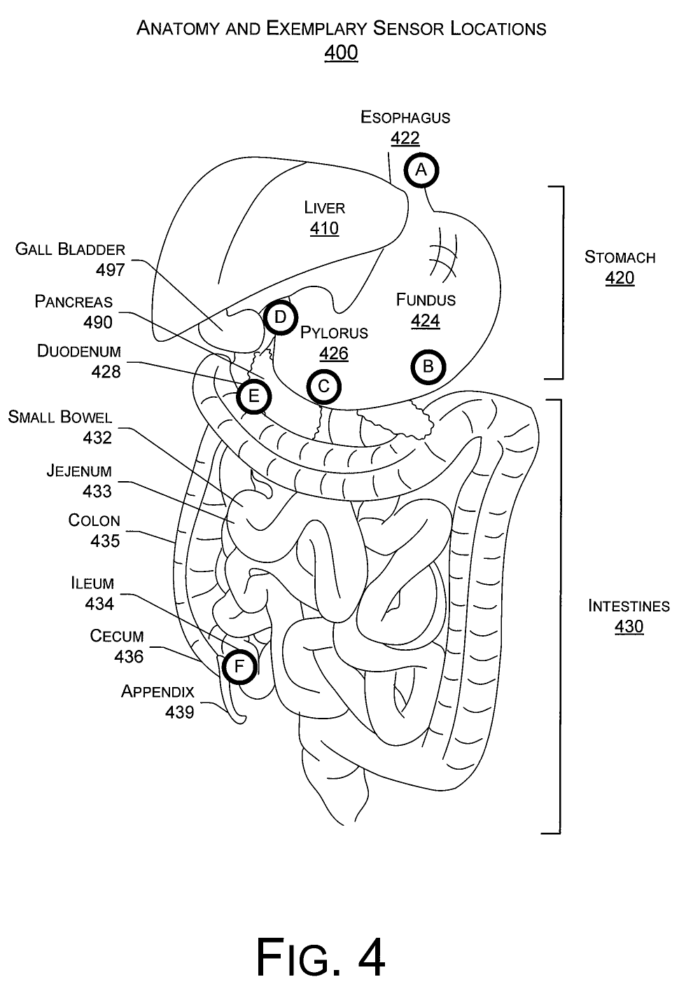

FIG. 4 shows an anatomical diagram for part of the digestive system along with exemplary sensor locations 400. The diagram 400 includes the liver 410, the stomach 420, the intestines 430, the pancreas 490 and the gall bladder 497. The stomach 420 includes labels that identify approximate locations of the esophagus 422, the fundus 424 and the pylorus 426. The intestines 430 include labels that identify approximate locations of the duodenum 428, the small bowel 432 (including the jejunum 433 and ileum 434) and the colon 435 (including the cecum 436). The appendix 439 is also identified.

Exemplary sensor locations, labeled A to F, include A (the esophagus 422), B (the fundus 424), C (the pylorus 426), D (the distal antrum), E (the duodenum 428) and F (the ileum 434/the cecum 436). As these portions of the gastrointestinal tract include muscles that can contract, a sensor may be positioned at one of these positions to measure contractions. For example, a sensor may be positioned to measure contractions of a particular portion of the gastrointestinal tract (e.g., a portion of the stomach). Where such a sensor further includes circuitry to measure VO₂, then contractions and VO₂ may be measured.

As already mentioned, a sensor may provide a measure or signal to an implantable device. In various examples, such an implantable device is configured to deliver GES. As an implantable device may be alternatively or additionally configured for autonomic nerve stimulation, a brief description of the vagus with respect to the digestive system follows. The vagus enters the abdomen with two trunks (the right, dorsal or posterior and the left, ventral or anterior) that track generally along the esophagus. When the vagi cross the diaphragm, in most individuals they divide into five distinctive branches: (i-ii) paired gastric branches (e.g., associated with the stomach and the proximal duodenum), (iii-iv) paired celiac branches (e.g., associated with duodenum, jejunum, ileum, cecum and colon) and (v) a single hepatic branch that originates from the ventral trunk (e.g., associated with distal antrum, duodenum, liver and gall bladder). With respect to the diagram 400 of FIG. 4, gastric braches of the anterior vagus include direct branches to the fundus 424 (V-direct), pyloric branches to the pylorus 426 (branches emanating from vagal supply to liver 410 that include superior pyloric nerves and inferior pyloric nerves), a hepatic branch or branches (V-hepatic) and the anterior nerve of Latarjet (V-Latarjet, principal anterior nerve of lesser curvature of the fundus 424). Again various branches are linked to the aforementioned Auerbach plexus and Meissner plexus.

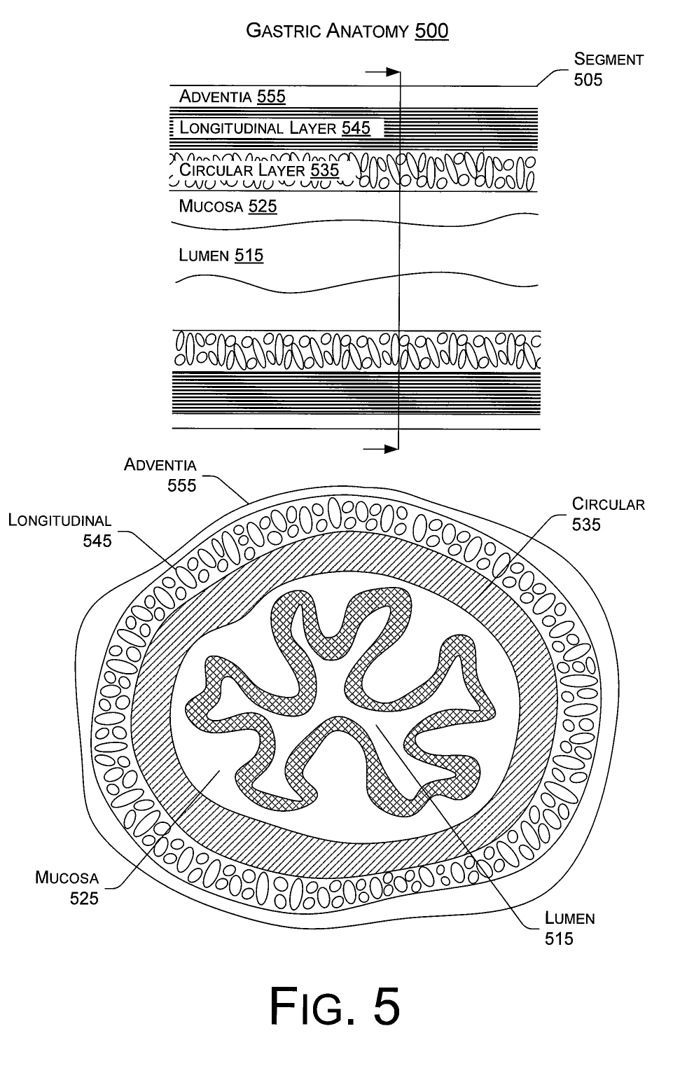

The various locations labeled A-F provide general guidance for sensor placement. With respect to specific guidance, FIG. 5 shows gastric anatomy 500 in two cross-sectional views of a segment 505 of the gastrointestinal tract. The gastrointestinal tract is essentially a tube extending from the oral cavity to the anus. This tube is organized into a series of four distinct layers which are fairly consistent throughout its length. Proceeding from the lumen 515 (i.e., abluminally from the lumen), the layers include:

Mucosa 525, which is the innermost layer (closest to the lumen 515) often described as the soft, squishy lining of the tract, consisting of epithelium, lamina propria and muscularis mucosae;

Muscularis circular 535, which is an inner circular layer of smooth muscle fibers wrapped around the long axis of the tract;

Muscularis longitudinal 545, which is an outer longitudinal layer of smooth muscle fibers extending parallel to the long axis of the tract; and

Adventitia/serosa 555, which is the outermost layer, which is called either adventitia (in regions where the tube passes through the body wall) or serosa (in regions where the tube passes through body cavities).

The muscularis of the stomach is often thicker than that elsewhere and the muscle fibers can be layered in more orientations (often described as assuming three layers, which are not readily distinguishable in routine sections). In the stomach, the inner layer of the muscularis forms a sphincter in the pyloric stomach (the pyloric sphincter).

Muscle contractions propel matter along the gastrointestinal tract. Muscle contractions of the gastrointestinal tract can be isolated to a single muscle layer or they may occur for multiple layers in a coordinated or uncoordinated manner. Contractions for multiple layers may occur in phase or out of phase. Phase locking may occur and contractions may be sequential, for example, where circular muscle contracts followed by longitudinal muscle. Contractions for multiple layers may be synchronous or asynchronous.

A study by Sarna (“Gastrointestinal longitudinal muscle contractions”, Am J Physiol Gastrointest Liver Physiol 265; G156-164, 1993) reported for a canine model, that, in the stomach, the longitudinal muscle contracted in a 1:1 relationship with the circular muscle contractions. Sarna noted that there was no significant difference between the frequency, duration and time of onset of gastric longitudinal and circular muscle contractions and their amplitudes were significantly correlated with each other. Sarna further noted that, in the small intestine when the circular muscle contracted, the longitudinal muscle exhibited passive elongation during the fasting and the fed state without any significant difference between the onset, duration and frequency of small intestinal circular muscle contractions and the passive longitudinal muscle elongations.

As described herein, an exemplary sensor can detect muscle contractions of the gastrointestinal tract and such contractions may indicate patient state or digestive phase (e.g., fasting state, fed state, cephalic phase, gastric phase, intestinal phase, etc.) and optionally degree of lumen occlusion.

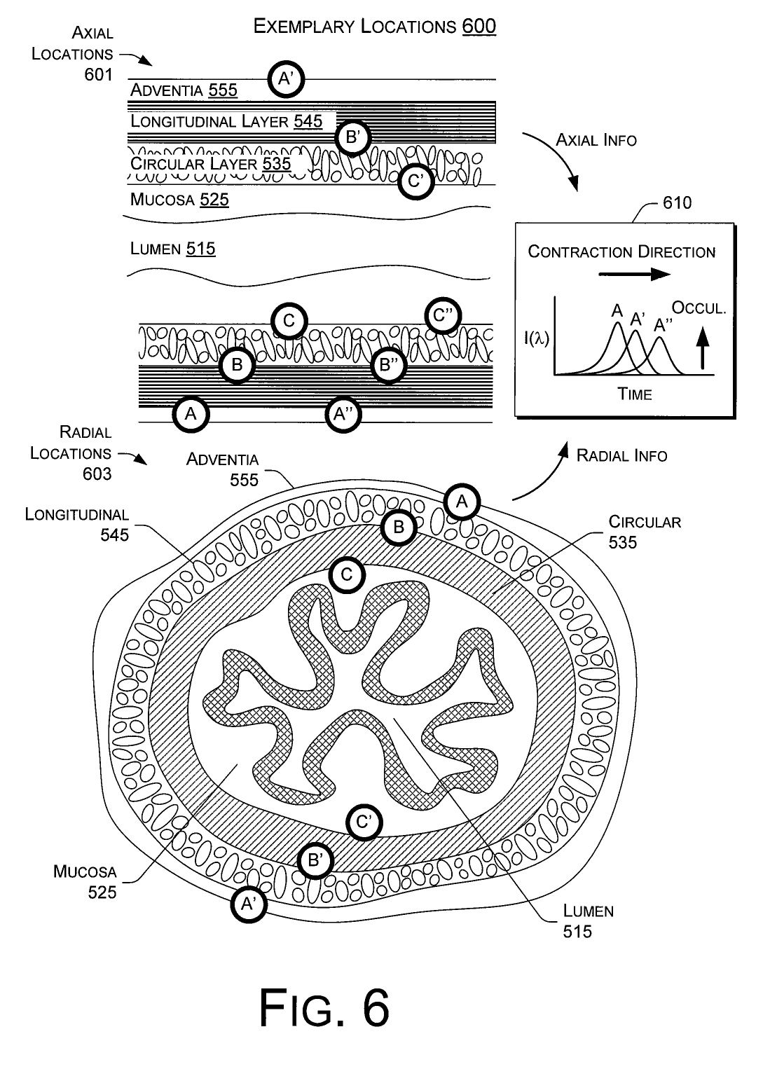

FIG. 6 shows some exemplary locations 600 for a sensor with respect to the anatomical diagram 500 of FIG. 5. As gastrointestinal tract anatomy varies, the exemplary locations 600 can be selected as appropriate to accommodate any variation. The locations 600 include axial locations 601 and radial locations 603. The axial locations 601 include locations A, A′ and A″ as in or adjacent the adventia; locations B, B′ and B″ as being approximately between the circular layer 535 and the longitudinal layer 545; and locations C, C′ and C″ as being in or adjacent the mucosa 525. The radial locations 603 include locations spaced apart by about 180 degrees. For example, a sensor may be positioned at location A and another sensor positioned at location A′ adjacent the gastrointestinal tract with an angle of about 180 degrees between the sensors. Such an arrangement can help to more accurately measure contractions and optionally degree of occlusion.

As explained, multiple sensors may be implanted into the body to measure gastrointestinal contractions. As indicated in a plot 610 of intensity versus time, the axial locations A, A′ and A″ can allow for detection of contraction direction and optionally degree of luminal occlusion. Specifically, axial placement can record time of maximum intensity (i.e., contraction) and a stronger contraction can be more white (i.e., less red) and thereby cause a larger increase in intensity (i.e., amplitude) versus a weaker contraction.

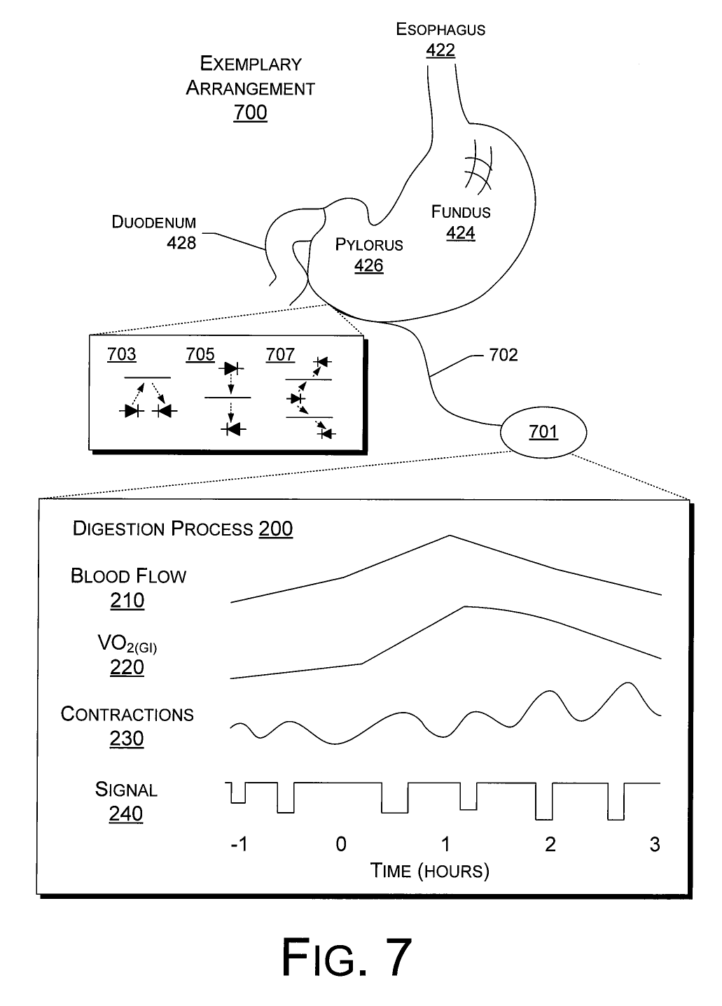

FIG. 7 shows an exemplary arrangement 700 for acquiring information about the digestion process 200, as explained with respect to FIG. 2. In FIG. 7, an implantable device 701 includes a lead 702 with one or more sensors configured, for example, according to sensor configuration 703, 705 or 707. The configuration 703 relies on an emitter to emit radiation and a detector to detect reflected radiation; the configuration 705 relies on an emitter to emit radiation and a detector to detect transmitted radiation; and the configuration 707 relies on an emitter to emit radiation and multiple detectors to detect the emitted radiation (e.g., reflected and/or transmitted).

Depending on features of the sensor, the implantable device 701 may acquire blood flow information 210, VO₂ information 220 and/or contraction information 230. A signal 240 represents a digital signal generated in response to contractions (noting that an analog signal may be used or an analog signal may be converted to a digital signal). In the example of FIG. 7, the signal 240 shows contractions with duration and amplitude information. A more simplified approach may rely on two digital states (e.g., 0 and 1) that represent a contraction or no contraction. Hardware and/or software techniques may be used to acquire any of a variety of signal that can be used to track contractions. The device 701 optionally stores acquired information 210, 220, 230 and/or signal 240 for any of a variety of purposes.

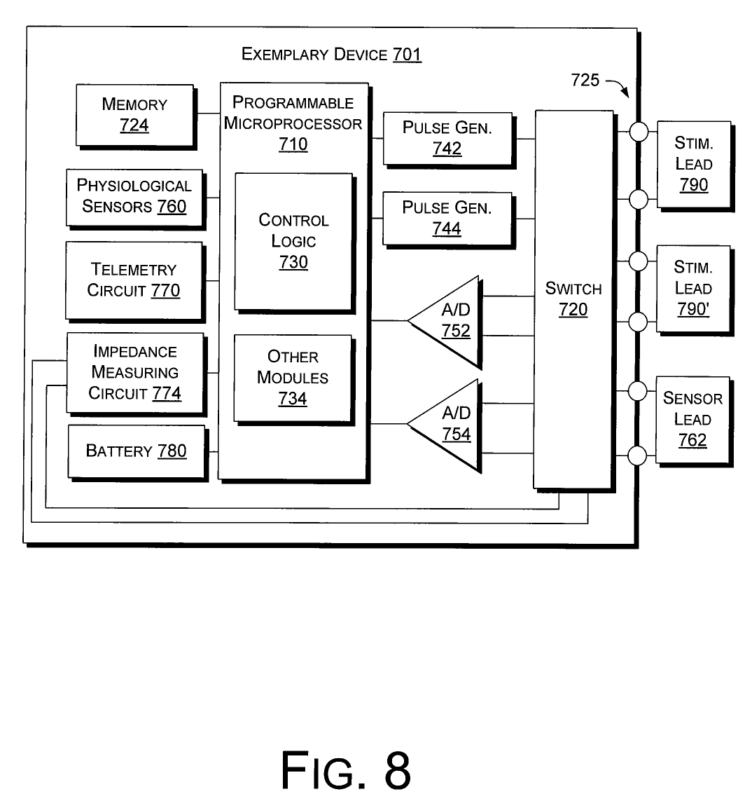

FIG. 8 shows a block diagram of the exemplary device 701. The device 701 may be optionally configured for sensing, activating and/or blocking activity of any number of organs, muscles and/or nerves. A basic device may include a processor, memory, one or more inputs, one or more outputs and control logic stored as instructions in the memory and operable in conjunction with the processor. The device 701 includes various additional features.

The exemplary device 701 includes a programmable microprocessor 710 that can implement control logic 730 and other instructional modules 734. Information may be stored in memory 724 and accessed by the programmable microprocessor 710. For delivery of energy, the device 701 includes one or more pulse generators 742, 744. The pulse generators 742, 744 may rely on a switch 720 for delivery of energy via one or more connectors 725. While a device may include one or more integral leads, in general, a device includes one or more connectors for connecting a lead or leads to the device. More particularly, the connectors 725 provide for electrically connecting one or more electrodes and/or one or more sensors to the circuitry of the device 701. In the example of FIG. 7, the switch 720 may select an appropriate electrode and/or sensing configuration. An electrode configuration may include an electrode from one lead and an electrode from another lead, a case electrode and another electrode or electrodes from a single lead.

The device 701 further includes one or more analog to digital converters 752, 754 for converting analog signals to digital signals or values. An exemplary sensor may provide a digital signal (e.g., high voltage as “1” and low voltage as “0”), for example, that corresponds to a contracted state and a relaxed state, respectively, of a portion of the gastrointestinal tract. The processor 710 may use a signal provided by one of the A/D converters 752, 754 to control a therapy or other process. A control signal from the processor 710 may instruct the switch 720 to select a particular electrode configuration for sensing electrical or other activity.

The device 701 may include one or more physiological sensors 760. Such sensors may be housed within a case of the device 701 (e.g., a motion sensor), include a surface mounted component, include a lead, include a remote sensor, etc. A sensor may provide a digital signal or an analog signal for use by the processor 710 or other circuitry of the device 701. A physiological sensor may provide a signal via one or more of the connectors 725 or it may be otherwise connected to device circuitry.

For purposes of communication with external or other implantable devices, the device 701 includes a telemetry circuit 770. The telemetry circuit 770 may include one or more antennae for transmission and/or receipt of electromagnetic signals. Such a circuit may operate according to a specialized frequency or frequencies designated for medical devices. Various conventional implantable devices rely on an associated programmer, which is typically an external computing device with a communication circuit suitable for communicating with an implantable device for purposes of data transfer, status checks, software download, etc. Where the circuit 770 communicates with an implantable device or a device in electrical connection with a patient's body, then the body may be a conductive medium for transfer of information. For example, the circuit 770 may be capable of communication with a specialized wristwatch where the body is relied upon as a conductor.

The device 701 further includes an impedance measuring circuit 774. Such a circuit may rely on instructions from the processor 710. For example, the processor 710 may instruct the circuit 774 to provide a measured impedance for a particular electrode configuration. In such an example, the processor 710 may also instruct the switch 720 to provide the circuit 774 with a particular electrode configuration. Impedance information may be used by the processor 710 for any of a variety of purposes (e.g., hardware condition, cardiac condition, edema, respiration, gastrointestinal, etc.). The processor 710 may store impedance or other information to memory 724 for later use or for transmission via the telemetry circuit 770.

The device 701 includes a power source, which is shown as a battery 780 in the example of FIG. 8. The battery 780 powers the processor 710 and optionally other circuitry, as appropriate. In general, the battery 780 provides power to the pulse generators 742, 744. Consequently, the battery 780 provides for operation of circuitry for processing control logic, etc., and provides for energy to activate tissue. A lead-based sensor may connect to the device 701 via one or more of the connectors 725 and be powered by the battery 780 (see, e.g., the lead 702 of FIG. 7). The battery 780 may be rechargeable, replaceable, etc.

In FIG. 8, the device 701 is shown as being connected to one or more stimulation leads 790, 790′ and a sensor lead 762. A stimulation lead may provide for GES and/or nerve stimulation. A sensor lead may be configured to delivery stimulation energy, as explained with respect to FIG. 3.

While the device 701 includes particular features, various exemplary devices, systems, methods, etc., may use or be implemented using a different device with more or less features.

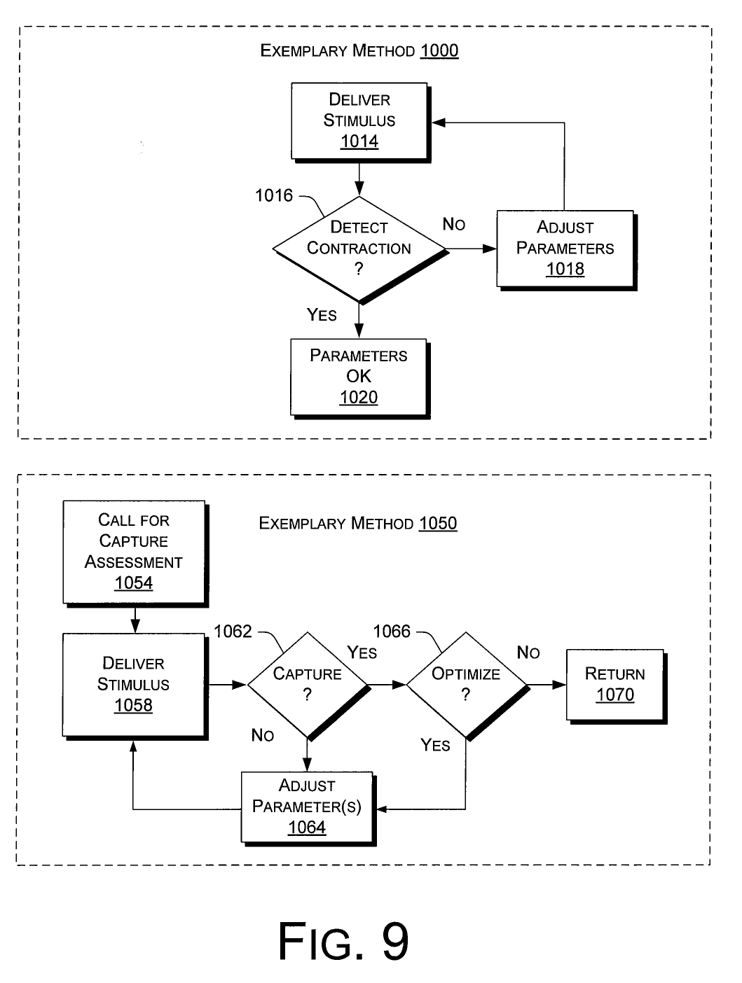

FIG. 9 shows an exemplary method 1000 for adjusting one or more stimulation parameters for GES and an exemplary method 1050 for assessing GES capture. The method 1000 is optionally implemented by an implantable device such as the device 701 of FIGS. 7 and 8. According to the method 1000, a delivery block 1014 delivers a stimulus to cause a gastrointestinal contraction. A decision block 1016 decides if the delivered stimulus caused a contraction. If the decision block 1016 decides that a contraction did not occur, then an adjustment block 1018 adjusts one or more stimulation parameters. However, if the decision block 1016 decides that a contraction did occur, then the method 1000 continues in block 1020 where the stimulation parameters are deemed acceptable for causing a gastrointestinal contraction. The method 1000 may be used to optimize energy expended for causing contractions. For example, the blocks 1014, 1016, 1018 and 1020 may form part of a loop used to minimize stimulation energy required to cause a gastrointestinal contraction. When implemented by an implantable device with a limited power supply, such a method can increase device longevity.

The method 1050 may be performed on a scheduled basis or in response to a condition such as the “no” branch of the decision block 1016 of the method 1000. The method 1050 commences in a call block 1054 that calls for capture assessment. In the example of FIG. 9, capture assessment includes responding to non-capture scenarios and optimizing one or more parameters associated with delivery of stimulation energy. Accordingly, a delivery block 1058 delivers energy to a portion of the gastrointestinal tract (e.g., a portion of the stomach). A decision block 1062 decides if a capture occurred in response to the delivered energy (e.g., based at least in part on information acquired from an exemplary sensor). If the decision block 1062 decides that capture did not occur (e.g., according to one or more criteria), then the method 1050 continues at an adjustment block 1064 to adjust one or more parameters associated with delivery of stimulation energy to a portion of the gastrointestinal tract. After adjustment, delivery of energy occurs per the delivery block 1058 and the loop continues until one or more exit criteria are met.

Referring again to the decision block 1062, if this block decides that capture occurred, then the method 1050 continues at another decision block 1066 that decides if optimization should occur to optimize delivery of energy (e.g., to conserve energy, to minimize adverse tissue response, etc.). If the decision block 1066 decides that optimization is not required (e.g., already at a minimum level of energy delivery, optimization occurred within the last X hours, etc.), then the method 1050 enters a return block 1070 to exit the capture assessment. However, if the decision block 1066 decides that optimization should occur, the method 1050 enters the adjustment block 1064, as already explained with respect to the “no” branch for the capture decision block 1062.

Information may be recorded for any of the exemplary methods described herein. For example, for the method 1050, information about capture, parameter values, optimization, etc., may be recorded. Recorded information may be examined by a clinician to assess patient health, operation of an implantable device, etc.

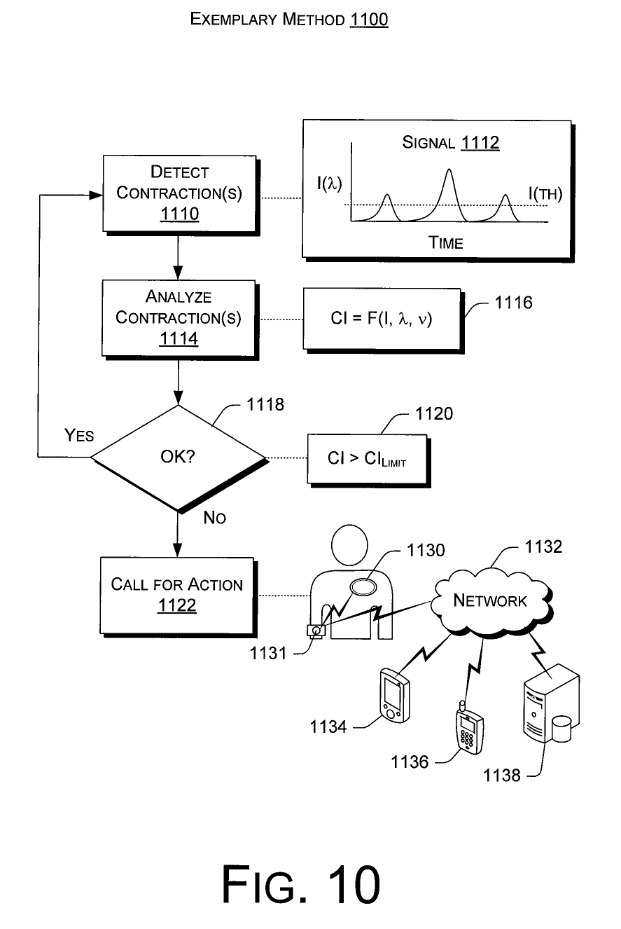

FIG. 10 shows an exemplary method 1100 for detecting gastrointestinal contractions. The method 1100 detects one or more contractions in a detection block 1110. For example, as indicated in the signal plot 1112, an exemplary sensor may detect a contraction when radiation intensity exceeds a certain intensity threshold, I(Th). An analysis block 1114 analyzes the one or more contractions, for example, using a formula or tables 1116. A formula may be used to analyze contraction information based on intensity, wavelength and frequency of contractions. According to the method 1100, a decision block 1118 decides, based at least in part on the analysis, if the contraction merits further action. For example, the analysis block 1114 may provide a value (e.g., contraction index “CI” 1116) and the decision block 1118 may compare the value to a limit (e.g., CI_{Limit} 1120). As shown in FIG. 10, if the value exceeds the limit, then no further action is taken and the method 1100 continues at the detection block 1110 where a subsequent contraction may be detected. However, if the value does not exceed the limit, then the method 1100 calls for action per a call block 1122.

In the example of FIG. 10, the call for action may be from an implantable device 1130 to an external device 1131 and/or to one or more devices 1134, 1136 and/or 1138 accessible by a network 1132. For example, the wristband device 1131 may receive a signal from the implantable device 1130 and, in turn, alert a care provider by dialing a cell phone number to reach the care provider's cell phone 1136. Where action is not urgent, information may be communicated to a computer 1134 or a database 1138.

An exemplary method may detect one or more contractions and, in response, call for a particular action. For example, certain contractions may indicate that a bowel movement is imminent (e.g., to occur within a few minutes). A sensor may sense such contractions and alert a patient. In the system of FIG. 10, the implantable device 1130 may alert a patient via the wearable device 1131. An alert may occur directly for some conditions and via review by a clinician for other conditions. For example, a clinician may receive patient information acquired by the implantable device 1130 and then issue an alert that alerts a patient via the wearable device 1131.

### CONCLUSION

Although exemplary mechanisms (e.g., implemented as or in methods, devices, systems, software, etc.) have been described in language specific to structural features and/or methodological acts, it is to be understood that the subject matter defined in the appended claims is not necessarily limited to the specific features or acts described.

1. An implantable device comprising:
an emitter to emit radiation to illuminate a portion of the stomach; and
a detector to detect emitted radiation reflected by the portion of the stomach wherein a contraction of the portion of the stomach alters the reflected radiation.

2. The device of claim 1 wherein the emitter emits radiation in response to an emitter control signal.

3. The device of claim 1 wherein the detector detects intensity of radiation reflected by the stomach.

4. The device of claim 3 wherein the detected intensity of radiation increases in response to a contraction of the stomach.

5. The device of claim 1 wherein the detector detects wavelength of radiation reflected by the portion of the stomach.

6. The device of claim 5 wherein the detected wavelength of radiation changes in response to a contraction of the portion of the stomach.

7. The device of claim 1 further comprising an output to output a signal related to detected radiation reflected by the portion of the stomach.

8. The device of claim 1 further comprising an anchor mechanism to anchor the device to the stomach.

9. The device of claim 1 further comprising a power supply to power the emitter and the detector.

10. An implantable device comprising:
an emitter to emit radiation to illuminate a portion of the stomach; and
a detector to detect emitted radiation transmitted by the portion of the stomach wherein a contraction of the portion of the stomach alters the transmitted radiation.

11. The device of claim 10 wherein the emitter emits radiation in response to an emitter control signal.

12. The device of claim 10 wherein the detector detects intensity of radiation transmitted by the portion of the stomach.

13. The device of claim 10 wherein the detector detects wavelength of radiation transmitted by the portion of the stomach.

14. The device of claim 10 further comprising an output to output a signal related to detected radiation transmitted by the portion of the stomach.

15. The device of claim 10 wherein a stomach tissue gap exists between the emitter and the detector.

16. The device of claim 10 further comprising an anchor mechanism to anchor the device to the stomach.

17. The device of claim 10 further comprising an anchor mechanism to anchor the emitter to the stomach and an anchor mechanism to anchor the detector to the stomach.

18. An implantable device comprising:
an emitter to emit radiation to illuminate a portion of the gastrointestinal tract;
a detector to detect emitted radiation reflected by the portion of the gastrointestinal tract or to detect emitted radiation transmitted by the portion of the gastrointestinal tract; and
wherein a contraction of the portion of the gastrointestinal tract alters the detected radiation.

19. The device of claim 18 wherein the detector detects an increase in reflected radiation during a contraction of the portion of the gastrointestinal tract or wherein the detector detects a change in transmitted radiation during a contraction of the portion of the gastrointestinal tract.

20. The device of claim 18 further comprising an anchor mechanism to anchor the device to the gastrointestinal tract.
